(19) United States
(12) Patent Application Publication
(10) Pub. No.: US 2025/0135075 A1
(43) Pub. Date: May 1, 2025

(54) FIBER-REINFORCED BIOCOMPOSITE MEDICAL IMPLANTS WITH DEFORMABLE PROTRUSIONS AND METHODS OF USE THEREOF

(71) Applicant: OSSIO LTD, Caesarea (IL)

(72) Inventors: Orahn PREISS-BLOOM, Caesarea (IL); Shay PARAG, Caesarea (IL); Tal ZEEVI, Caesarea (IL)

(21) Appl. No.: 18/571,280

(22) PCT Filed: Jul. 4, 2022
(86) PCT No.: PCT/IL2022/050711

§ 371 Date: Dec. 18, 2023

Related U.S. Application Data
(60) Provisional application No. 63/223,152, filed on Jul. 19, 2021.

(51) Int. Cl. A61L 31/12; A61B 17/00; A61B 17/72; A61L 31/14

(52) U.S. Cl. CPC A61L 31/125; A61B 17/72; A61L 31/148; A61B 2017/00004; A61L 2430/02

(57) ABSTRACT

Biocomposite materials which overcome the drawbacks of the background art. Medical implants are provided that incorporate novel structures, alignments, orientations and forms comprised of such surface treated bioabsorbable materials, such as for example implants featuring protrusions.

### FIELD OF THE INVENTION

The present invention is to biocomposite medical implants comprising deformable protrusions to increase pull out strength and methods of use thereof, and in particular to such material, implants and methods of treatment that have medical applications.

### BACKGROUND OF THE INVENTION

The mechanical strength and modulus (approximately 3-5 GPa) of non-reinforced resorbable polymers is insufficient to support fractured cortical bone, which has an elastic modulus in the range of approximately 15-20 GPa. For example, in an article the bending modulus of human tibial bone was measured to be about 17.5 GPa (Snyder S M Schneider E, Journal of Orthopedic Research, Vol. 9, 1991, pp. 422-431). Therefore, the indications of existing medical implants constructed from resorbable polymers are limited and their fixation usually requires protection from motion or significant loading. These devices are currently only a consideration when fixation of low stress areas is needed (i.e. non-load bearing applications) such as in pediatric patients or in medial malleolar fractures, syndesmotic fixation, maxillofacial, or osteochondral fractures in adults.

A new class of reinforced composite biomaterials (biocomposites) has been recently introduced wherein a bioabsorbable and biocompatible polymer is reinforced by bioabsorbable, biocompatible glass fibers. These materials can achieve improved mechanical properties. These materials also involve a compatibilizer to bind the polymer to the reinforcing fibers. Examples of such materials are described in the following two patent applications, which are included fully herein by reference as if fully set forth herein:
1. Biocompatible composite and its use (WO2010122098)
2. Resorbable and biocompatible fiber glass compositions and their uses (WO2010122019)

These materials have been further described and characterized in publications associated with these patents including
1. Lehtonen T J et al. Acta Biomaterialia 9 (2013) 4868-4877
2. Lehtonen T J et al. J Mech Behavior BioMed Materials. 20 (2013) 376-386

The development of this class of materials described in the background art has focused on the composition of the materials: the bioabsorbable polymer, the reinforcing mineral fiber, the compatibilizer, and the combinations between them. These compositions have been demonstrated to be capable of achieving mechanical properties superior to the mechanical properties previously achieved with bioabsorbable polymers alone.

However, while material composition is one parameter that can affect mechanical properties of a medical implant, when it comes to composite materials, the material composition does not by itself ensure mechanical properties that are sufficient for the implant to achieve its desired biomechanical function. In fact, reinforced composite medical implants with identical compositions and identical geometries can have vastly different mechanical properties. Furthermore, even within the same implant, mechanical properties can vary greatly between different mechanical axes and between different types of mechanical strength measurements.

Various deficiencies exist in many of the commercially available implants and there exists a need for improved implants to address these deficiencies.

### SUMMARY OF THE INVENTION

The background art does not teach or suggest biocomposite materials that have one or more desirable mechanical characteristics. The background art also does not teach or suggest such materials that can achieve a desired biomechanical function.

The present invention, in at least some embodiments, relates to biocomposite materials which overcome the drawbacks of the background art. According to at least some embodiments, medical implants are provided that incorporate novel structures, alignments, orientations and forms comprised of such surface treated bioabsorbable materials, such as for example implants featuring protrusions.

Unless otherwise defined, all technical and scientific terms used herein have the same meaning as commonly understood by one of ordinary skill in the art to which this invention belongs. The materials, methods, and examples provided herein are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1, shows a 4.0 mm×50 mm ribbed hexagonal nail with an edge to edge implant diameter of 3.4 mm and an edge to edge protrusion diameter of 3.9 mm with a protrusion height of 0.25 mm. FIG. 1A and FIG. 1C show the nail structure, FIG. 1B shows the nail from a different angle, FIG. 1D shows a cross-section of the nail, FIG. 1E-FIG. 1H show detailed views of the protrusions.

FIG. 2, shows a 2.4 mm×50 mm ribbed hexagonal nail with an edge to edge implant diameter of 2.0 mm and an edge to edge protrusion diameter of 2.5 mm with a protrusion height of 0.25 mm. FIG. 2A and FIG. 2C show the nail structure, FIG. 2B shows the nail from a different angle, FIG. 2D shows a cross-section of the nail, FIG. 2E-FIG. 2H show detailed views of the protrusions.

FIG. 3, shows a 4.0 mm×50 mm compression screw with an edge to edge implant diameter of 3.0 mm and an edge to edge protrusion diameter of 4.0 mm with a protrusion height of 0.5 mm. FIG. 3A and FIG. 3C show the screw structure, FIG. 3B shows the screw from a different angle, FIG. 3D shows a cross-section of the screw, FIG. 3E-FIG. 3F show detailed views of the protrusions.

FIG. 4, shows a 4.0 mm×70 mm ribbed hexagonal nail with an edge to edge implant diameter of 3.4 mm and an edge to edge protrusion diameter of 3.9 mm with a protrusion height of 0.25 mm. FIG. 4A and FIG. 4C show the nail structure, FIG. 4B shows the nail from a different angle, FIG. 4D shows a cross-section of the nail, FIG. 4B-FIG. 4H show detailed views of the protrusions.

FIG. 5, shows a ribbed nail prior to insertion in a pre-drilled hole in the bone of a subject (FIG. 5A) and a ribbed nail partially inserted in a pre-drilled hole in the bone of a subject (FIG. 5B). In the expanded views of the protrusions it is shown in the inserted segment of the nail, the ribs deform slightly as the implant is inserted into the bone tunnel.

FIG. 6 shows a side view cut out of a ribbed nail to show the drill hole (the white bole) with the ribs (shown below in black). The ribs of the 2.4 mm ribbed nail are expected to form an interference fit with the 2.4 mm drill hole (the white bole) with the ribs providing interference with the hole at the bone interference area (shown below in grey). Preferably, the bone tunnel diameter is larger than the core diameter of the implant but smaller than the diameter of the ribs.

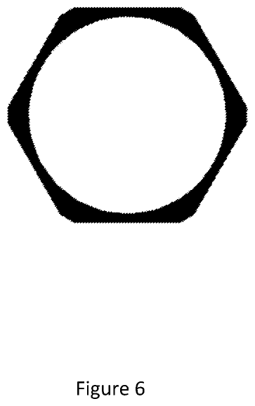

### DETAILED DESCRIPTION OF SOME EMBODIMENTS

A medical implant according to at least some embodiments of the present invention is suitable for load-bearing orthopedic implant applications and comprises one or more bioabsorbable materials where sustained mechanical strength and stiffness are critical for proper implant function.

According to at least some embodiments of the present invention, there is provided orthopedic implants, such as those for bone fixation, made from reinforced bioabsorbable composite materials. Specifically, implants according to at least some embodiments incorporate characteristics, features, or properties that can either only be achieved using the reinforced bioabsorbable composite materials or are specifically advantageous for implants comprised of these types of materials, or optionally a combination of both in a single implant.

The medical implants of the present invention have unique mechanical properties. They have great clinical benefit in that these implants can have mechanical properties that are significant greater than those of the currently available bioabsorbable polymer implants. The term “mechanical properties” as described herein may optionally include one or more of elastic modulus, tensile modulus, compression modulus, shear modulus, bending moment, moment of inertia, bending strength, torsion strength, shear strength, impact strength, compressive strength and/or tensile strength.

Any of the embodiments or sub-embodiments as described herein may be combined, for example in regard to any of the implant properties, implant structures or implant surface treatments, or any combination of any aspect of the same.

Without wishing to be limited by a closed list or a single hypothesis, the biocomposite implants described herein represent a significant benefit over metal or other permanent implants (including non absorbable polymer and reinforced polymer or composite implants) in that they are absorbable by the body of the subject receiving same, and thus the implant is expected to degrade in the body following implantation. Again without wishing to be limited by a closed list or a single hypothesis, they also represent a significant benefit over prior absorbable implants since they are stronger and stiffer than non-reinforced absorbable polymer implants in at least one mechanical axis. In fact, these reinforced composite polymer materials can even approach the strength and stiffness of cortical bone, making them the first absorbable materials for use in load bearing orthopedic implant applications.

On an underlying level, there is a great difference between the reinforced biocomposite implants and previous implants from metal, plastic, and other traditional medical implant materials. Traditional medical implant materials are isotropic such that their mechanical properties are identical in all axes. This simplifies implant design as the mechanical strength of the implant is determined solely based on the geometry of the implant and the inherent material properties of the material. Without wishing to be limited by a closed list or a single hypothesis, for reinforced biocomposite implants, the inherent material properties of the biocomposite (i.e. the biocomposite in amorphous or non-aligned form) are actually quite low and can approximate the mechanical properties of the polymer alone. As such, implant geometries for implants constructed from these biocomposite materials does not inherently determine implants that are mechanically strong or stiff.

However, the medical implants of the present invention in at least some embodiments are able to exceed the mechanical properties of previous bioabsorbable implants, including previous biocomposite implants in one or more mechanical axes and in one or more mechanical parameters. Preferably these implants feature structures and forms in which the reinforcing fibers are aligned within the implant in order to provide the implant load bearing strength and stiffness in the axes in which these properties are biomechanically required. Thus, either the entire implant or segments of the implant are anisotropic (i.e. they have different mechanical properties in different axes). With these anisotropic implants, the implant mechanical design cannot rely solely on the geometry of each part. Rather, the specific alignment of the reinforcing fibers within the implant and the resulting anisotropic mechanical profile are a key parameter in determining the biomechanical function of the implant.

Aside from the mechanical considerations related to the anisotropic medical implants, there are additional limitations in that medical implants using these reinforced biocomposite materials cannot be designed according to existing implant designs due to the limitations associated with producing parts from these composite materials.

For example, metal implants or permanent polymer implants may be produced by machining. Even fiber-reinforced permanent polymer implants may be machined without adversely affecting the mechanical properties. However, absorbable, reinforced composite material implants cannot be machined without causing damage to the underlying material since machining will expose reinforcing fibers from the polymer, thus causing their strength to degrade quickly once they are directly exposed to body fluid following implantation.

At the other end of the spectrum, pure polymer or very short (<4 mm) fiber-reinforced polymer implants may be manufactured using straightforward injection molding processes. Injection molding of these materials does not, however, result in sufficiently strong implants. Therefore, specialized designs and production methods are required in order to design and produce an implant that can benefit from the superior mechanical properties of the previously described reinforced bioabsorbable composite materials.

### Definitions

By “biocomposite material” it is meant a composite material that is biologically compatible or suitable, and/or which can be brought into contact with biological tissues and/or which can be implanted into biological materials and/or which will degrade, resorb or absorb following such implantation.

By “biocompatible” it is meant a material that is biologically compatible or suitable, and/or which can be brought into contact with biological tissues, and/or which can be implanted into biological materials.

By “surface treated” biocomposite material it is meant a material which features at least a surface layer, and optionally a plurality of surface layers.

The term “biodegradable” as used herein also refers to materials that are degradable, resorbable or absorbable in the body. “Biodegradable” as used herein is a generalized term that includes materials, for example polymers, which break down due to degradation with dispersion in vivo. The decrease in mass of the biodegradable material within the body may be the result of a passive process, which is catalyzed by the physicochemical conditions (e.g. humidity, pH value) within the host tissue. In a preferred embodiment of biodegradable, the decrease in mass of the biodegradable material within the body may also be eliminated through natural pathways either because of simple filtration of degradation by-products or after the material's metabolism (“Bioresorption” or “Bioabsorption”). In either case, the decrease in mass may result in a partial or total elimination of the initial foreign material. In a preferred embodiment, said biodegradable composite comprises a biodegradable polymer that undergoes a chain cleavage due to macromolecular degradation in an aqueous environment.

A polymer is “absorbable” as described herein if it is capable of breaking down into small, non-toxic segments which can be metabolized or eliminated from the body without harm. Generally, absorbable polymers swell, hydrolyze, and degrade upon exposure to bodily tissue, resulting in a significant weight loss. The hydrolysis reaction may be enzymatically catalyzed in some cases. Complete bioabsorption, i.e. complete weight loss, may take some time, although preferably complete bioabsorption occurs within 24 months, most preferably within 12 months.

The term “polymer degradation” means a decrease in the molecular weight of the respective polymer. With respect to the polymers, which are preferably used within the scope of the present invention said degradation is induced by free water due to the cleavage of ester bonds. The degradation of the polymers as for example used in the biomaterial as described in the examples follows the principle of bulk erosion. Thereby a continuous decrease in molecular weight precedes a highly pronounced mass loss. Such loss of mass is attributed to the solubility of the degradation products. Methods for determination of water induced polymer degradation are well known in the art such as titration of the degradation products, viscometry, differential scanning calorimetry (DSC).

Bulk degradation refers to a process of degradation in which there is at least some perfusion of fluid through the material that is being degraded, such as the body of the implant, thereby potentially degrading the bulk of the material of the implant (as opposed to the external surface alone). This process has many effects. Without wishing to be limited to a closed list, such bulk degradation means that simply making an implant larger or thicker may not result in improved retained strength.

Surface degradation refers to a process of degradation in which the external surface undergoes degradation. However, if there is little or no perfusion of fluid through the material that is being degraded, then the portion of the implant that is not on the surface is expected to have improved retained strength over implants in which such perfusion occurs or occurs more extensively.

The term “load bearing” optionally also includes partially load bearing. According to various embodiments, the load bearing nature of the device (implant) may optionally include flexural strengths above 200 MPa, preferably above 300 MPa, more preferably above 400 MPa, 500 MPa, and most preferably above 600 MPa or any integral value in between.

The term “cannulated” means the interior of the implant is hollow to form a tube like shape.

Biocomposite medical implants have been previously described in WO 2016/035088. WO 2016/035089, WO 2016/103049, WO 2017/155956, WO 2018/002917, WO2019/049062, WO 2019/123462 and WO2020/0044327 the entire contents of each of which are incorporated herein by reference.

In continuous fiber-reinforced biocomposite implants, the fiber reinforcement can provide mechanical properties with high strength and stiffness. For example, medical implants with flexural modulus in excess of 10 GPa. These properties can be very beneficial for the overall biomechanical function of the implant in resisting flexural deformation under physiological load.

As previously described in WO 2018/002917, a high ratio of mineral fiber content, for example in the range of 40-70% can contribute to mechanical properties such as high strength and modulus. These mechanical properties are established by the underlying core structure, hereafter referred to as “core” of the medical implant.

It has now been discovered that one or more novel protrusions along the length of the implant allow the implant to lodge itself firmly within a drill hole or drill tunnel in the bone.

According to at least some embodiments, the reinforced biocomposite medical implant of the present invention is comprised of an internal composition region, or “body” or “core” and a surface “region” or “layer”, defined as the region comprising the surface layer of part or all of the implant.

### Surface Layer

In one embodiment, the inner surface layer composition is different than an outer surface layer composition (as described in WO 2017/155956 and WO2020/0044327). In one embodiment, the surface layer is a uniform polymer or a bio composite composition different than the internal composition.

In one embodiment, the implant is a mineral fiber-reinforced biocomposite implant and fewer reinforcing fibers are present in either the entire surface region or the outermost surface region as compared with the internal composition region.

In one embodiment, the surface region covers the entire surface of the implant. In one embodiment, the surface region covers a percentage of the surface of the implant, with the remaining surface being of the same properties as the internal composition region. In one embodiment, the surface region covers more than 50% of the entire surface of the implant.

In one embodiment the surface layer may have various thicknesses, of which various non-limiting examples are given herein. For example, the surface layer is defined as an outer layer of up to 100 micron depth. In one embodiment, said outer layer is up to 50 micron depth. In one embodiment, said outer layer is up to 20 micron depth. In one embodiment, said outer layer is up to 5 micron depth. In one embodiment, the thickness of the polymer surface is not more than 100 microns, 90, 80, 70, 60, 50, 40, 30, 20 or anything in between. In one embodiment, the surface region can be defined as a layer of average depth in the range of 0.1-200 micron, preferably 1-100 micron, more preferably 2-75 micron and most preferably 5-50 micron.

In one embodiment, the surface layer varies across different cross-sections of the medical implant. In one embodiment, the depth of the surface layer is in the range of 1-50 microns in one cross-section of the implant and greater than 50 microns in another cross section. In one embodiment, the depth of the surface is in the range of 5-50 microns in one cross-section of the implant and greater than 100 microns in another cross section.

In one embodiment the implants of the invention comprise various surface layer structures. For example, said surface layer comprises a plurality of separate distinguished layers, each comprising a different composition.

In one embodiment, the surface region or layer may be further broken down into an outermost (external) surface region and innermost (internal) surface region, each of which may have different properties. In one embodiment the surface layer comprises an inner surface layer and an outer surface layer.

In one embodiment, the outer layer has an average depth in the range of 0.1-100 micron, preferably 0.5-50 micron, more preferably 1-25 micron and most preferably 1-10 micron. In one embodiment, the inner layer has an average depth in the range of 0.1-100 micron, preferably 0.5-50 micron, more preferably 1-25 micron and most preferably 1-10 micron. In one embodiment in the implants of the invention the surface layer comprises at least an inner layer and an outer layer, wherein the outer layer is up to 3 microns and the inner layer is up to 20 microns.

### Surface Layer Mineral Content

In one embodiment, the surface region has lower mineral content than the internal composition region (as described in WO 2017/155956 and WO2020/0044327).

In one embodiment, the innermost surface region has:
Sodium (Na) weight composition of less than 1.9%, preferably less than 1.5%. Preferably the sodium weight composition of innermost surface region is 50% less than sodium weight composition of internal composition and more preferably 30% less.
Magnesium (Mg) weight composition of less than 0.3%, preferably less than 0.2%. Preferably the magnesium weight composition of innermost surface region is 50% less than magnesium weight composition of internal composition and more preferably 30% less.
Silica (Si) weight composition of less than 6%, preferably less than 4%. Preferably silica weight composition of innermost surface region is 50% less than silica weight composition of internal composition and more preferably 30% less.
Phosphorous (P) weight composition of less than 3%, preferably less than 1%.
Calcium (Ca) weight composition of less than 1%, preferably less than 0.5%. Preferably calcium weight composition of innermost surface region is 50% less than calcium weight composition of internal composition and more preferably 30% less.

In one embodiment, the outermost surface region has higher mineral content than the innermost surface region.

In one embodiment, outermost surface region has:
Sodium (Na) weight composition of less than 1.9%, preferably less than 1.5%
Magnesium (Mg) weight composition of less than 1%, preferably less than Preferably magnesium weight composition of outermost surface region is greater than magnesium weight composition of innermost surface region.
Silica (Si) weight composition of less than 6%, preferably less than 4%. Preferably silica weight composition of outermost surface region is 50% less than silica weight composition of internal composition and more preferably 30% less.
Phosphorous (P) weight composition in range of 1-15%, preferably 3-13%. Preferably phosphorous weight composition of outermost surface region is at least 50% greater than phosphorous weight composition of innermost layer or than internal composition or than both; more preferably at least 70% greater and most preferably at least 90% greater.
Calcium (Ca) weight composition in range of 15-50%, preferably IS-30%. Preferably calcium weight composition of outermost surface region is at least 100% greater than calcium weight composition of innermost layer, more preferably at least 500% greater and most preferably at least 1000% greater.

### Protrusions

In one embodiment, the implants of the present invention comprise one or more protrusions along the length of the implant surface. These protrusions protrude out from the implant and may be of any suitable shape including but not limited to ribs, threads, hooks, quills, spikes, burrs, or clips etc., or combination thereof.

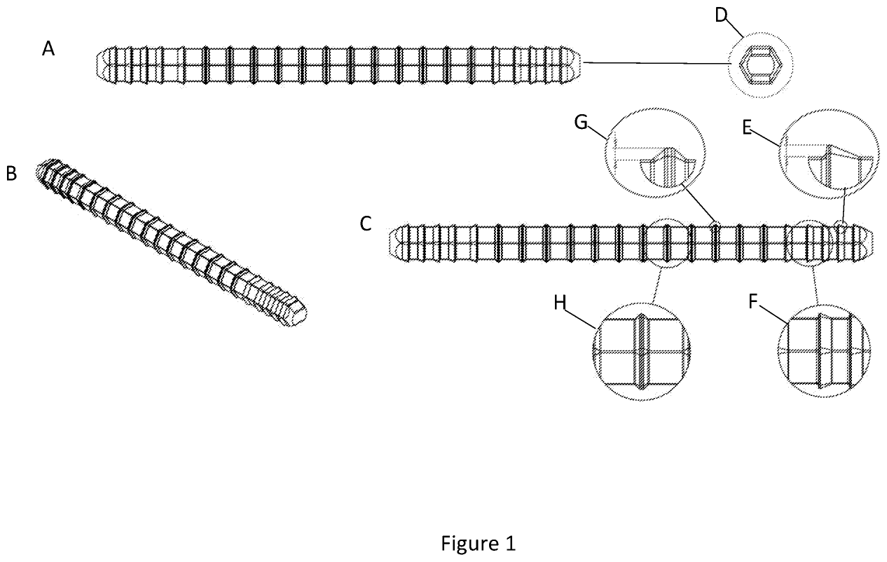

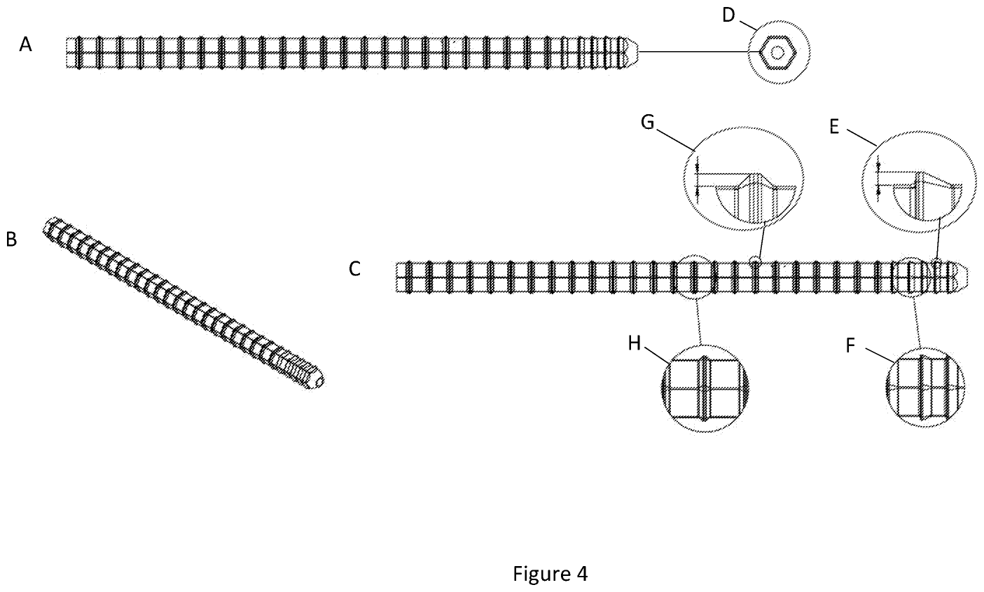

FIGS. 1-4 show non-limiting examples of suitable shapes of the protrusions or “ribs” suitable for use in the implants of the present invention. As shown in FIGS. 1-4, E-H show detailed examples of suitable ribs for use in the implants of the present inventions. In one embodiment, E and F show unidirectional ribs, that is, they are intended to be inserted in a specific direction and embed themselves in that direction. In one embodiment, G and H show bidirectional ribs, that is, they can be inserted from either direction and be embedded from either direction.

In one embodiment of the present invention, the implant can be entirely unidirectional or can have two directions and cut in the middle so both of the sides can be inserted.

In one embodiment, the protrusions are between 0.10 mm and 2 mm, 0.2 mm and 1 mm or 0.4 and 0.8 mm in width at the base where the protrusion meets the implant.

In one embodiment, the protrusions are between 0.01 mm and 0.3 mm, 0.02 mm and 0.2 mm or 0.02 and 0.1 mm in width at the tip.

In one embodiment, the protrusions are between 0.05 mm and 2 mm, 0.1 mm and 1 mm or 0.2 and 0.8 mm in height.

Optionally, the height of the protrusions may be considered in regard to the diameter, or alternatively the height or width of the implant. In some embodiments, the height or width are selected according to what is larger. In other embodiments the height or width are selected according to what is smaller. Diameter, height and width of the implant are described herein as “the measured dimension of the implant”. The height of the protrusions is optionally in a ratio to the measured dimension of the implant, in a range of 1:40 to 40:1; 1:30 to 30:1, 1:20 to 20:1, 1:10 to 10:1; 1:8 to 8:1; a range of 1:5 to 5:1; a range of 1:4 to 4:1; a range of 1:3 to 3:1, a range of 1:2 to 2:1, or any integral value in between.

In one embodiment, the protrusions for an angle with the implant surface of between 50° and 160°, 70° and 1502, 80° and 145°, or 90° and 140°.

In one embodiment, the protrusions increase the resistance to the implant being pulled out of the tunnel, as used herein, “pull-out force” or “pull-out resistance”.

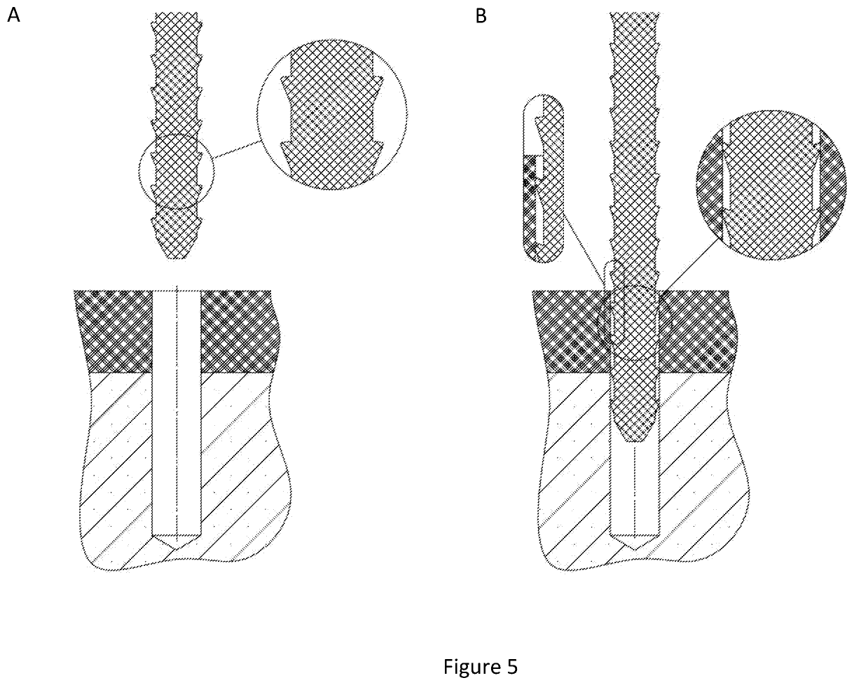

In one embodiment, when the medical implant of the present invention is implanted into a pre-drilled hole in the bone of a subject, the protrusions increase the pull-out resistance by undergoing deformation as the implant is inserted into the tunnel. The deformation can be elastic deformation, plastic deformation, or a combination of both. In one embodiment, the deformation of the protrusion in the axis perpendicular to the axis of insertion of the medical implant creates an increased frictional force that resists pull-out of the implant in the reverse direction of insertion. This principle is illustrated in FIG. 5. In FIG. 5(B) the tips of the protrusions are deformed from the insertion into the pre-drilled hole in the bone. The increased frictional force exerted by the deformed ribs on the bone walls increases pull out resistance.

Without wishing to be limited by a closed list, a number of factors may contribute to an increase in force necessary to pull the implant from the bone, which is a desired outcome as it increases the security of anchoring. The elastic component of the deformation of the protrusions may create a force from the protrusions pushing against the bone tunnel wall. This force orthogonal to the direction of implant pull-out thus increases the required frictional force to pull the implant out from the bone tunnel. The protrusions may also lodge themselves into the sides of the bone tunnel by their unidirectional geometry such that they create an interference fit within the bone and become very difficult to pull out.

It has also been discovered that, the high mineral content of the core which is responsible for the high strength and modulus of the overall implant may not be appropriate for the implant's protrusions. High mineral content can result in brittleness. If the protrusions are brittle, to the extent that they may break when they are deformed upon implant insertion, then they will fail to serve their purpose of increasing pull-out resistance.

In one embodiment, the present invention is a continuous fiber reinforced medical implant comprising one or more protrusions that is reinforced with a high percentage of mineral fibers for improved mechanical properties and further where the mineral content percentage of its protrusions is lower than the mineral content of the core of the implant.

### Protrusion Mineral Content

Each of the core body and the protrusions could have a surface layer. The mineral content of those surface layers are not necessarily related to the mineral content in the body or protrusions themselves. i.e. the surface layer content is independent of the body/protrusions content.

Preferably the average mineral content of the protrusions is in the range of 0-30%. More preferably the range is 10-30%. Even more preferably 15-25%.

Preferably the average mineral content of the protrusions as a percentage of their entire composition is 10-40 percentage points lower than the average mineral content of the core as a percentage of its entire composition; more preferably 15-35 percentage points lower, and most preferably 20-35 percentage points lower.

When referring to the individual mineral content of each mineral as a weight percentage of the entire protrusion, the individual mineral content ranges are preferably (in % of total protrusion composition): Na₂O: 1.1-5.7, CaO: 0.8-4.2, MgO: 0.15-2.4, B₂O₃: 0.05-0.9, SiO₂: 6.6-21.9.

### Core Mineral Content

Preferably the average mineral content of the medical implant core or body is in the range of 35-75%. More preferably the range is 40-70%. Even more preferably 40-60% and most preferably 45-60%.

In one embodiment, the mineral content of the mineral fibers comprises, all mol %: Na₂O: 11.0-19.0, CaO: 9.0-14.0, MgO: 1.5-8.0, B₂O₃: 0.5-3.0, Al₂O₃: 0-0.8, PO₃: 0.1-0.8, SiO₂: 67-73. Optionally, the mineral material of said body composition comprises ranges of the following elements, all mol %: Na₂O: 12.0-13.0 mol. %, CaO: 9.0-10.0 mol. %, MgO: 7.0-8.0 mol. % B₂O₃: 1.4-2.0 mol. % P₂O₃: 0.5-0.8 mol. %, SiO₂: 68-70 mol. %. Optionally, the mineral material of said body composition comprises ranges of the following elements, all mol %: Na₂O: 11.0-19.0, CaO: 8.0-14.0, MgO: 2-8.0, B₂O₃: 1-3.0, Al₂O₃: 0-0.5, P₂O₃: 1-2, SiO₂: 66-70% mol %.

In one embodiment, the individual mineral content of each mineral as a weight percentage of the entire core, the individual mineral content ranges are preferably (in % of total core composition): Na₂O: 4.4-11.4, CaO: 3.2-8.4, MgO: 0.6-4.8, B₂O₃: 0.2-1.8, SiO₂: 26.4-43.8.

### Dimensions and Shapes

In one embodiment, the cross-section of the implants of the present invention can be of any suitable shape including but not limited to circle, oval, triangle, square, rectangle, pentagonal, hexagonal, heptagonal, octagonal etc.

In one embodiment, the diameter of the implants of the present invention is between 1 man and 14 mm, 1 mm and 10 mm, 1.5 mm and 6 mm, or 2.0 mm and 4 mm.

In one embodiment, the diameter of the implant is measured from the tip of one protrusion to the tip of the opposite protrusion, as used herein, the “protrusion diameter”. In one embodiment, the diameter of the implant is measured from the outer edge of the surface layer to the opposite outer edge of the surface layer, not including the protrusions, as used herein the “implant diameter”.

In one embodiment, the implant diameter of the implants of the present invention is between 1 mm and 14 mm, 1 mm and 10 mm, 1.5 mm and 6 mm, or 2.0 mm and 4 mm.

In one embodiment, the protrusion diameter of the implants of the present invention is between 1 mm and 14 mm, 1 mm and 10 mm, 1.5 mm and 6 mm, or 2.0 mm and 4 mm.

In one embodiment, the length of the implant is between 10 mm and 200 mm, or 15 mm and 150 mm.

In one embodiment the implant diameter is smaller than the drill hole diameter and the protrusions extend past the implant diameter. In one embodiment the outer-rib diameter is larger than the drill hole.

In one embodiment, one or more cannulation or screw bole voids may be present on the inside of implant. In one embodiment, the implants of the present invention are cannulated.

### Surface Treated

The surface layer may optionally be implemented with a variety of different portions of the surface area having a different composition than the body composition as described in WO 2017/155956 and WO2020/044327 the entire contents of which are incorporate herein by reference in their entirety. For example and without limitation, optionally more than 10% of an area of the surface is of a different composition than the body. Optionally more than 30% of the surface area is of a different composition than the body. Optionally more than 50% of the surface area is of a different composition than the body.

Optionally the treated portion of the surface layer has a roughness increase by more than five times the untreated surface. Optionally the treated portion of the surface layer has a roughness increase by more than ten times the untreated surface. Optionally the treated surface area increases the surface area by more than 15%. Optionally the treated surface area increases the surface area by more than 50%. Optionally the surface has a different mineral composition.

In one embodiment in the implants of the invention the surface layer comprises at least an inner layer and an outer layer, wherein the outer layer is up to 3 microns and features an increase in phosphate to more than 5% w/w, wherein the inner layer is up to 20 microns and does not feature phosphate.

Optionally a surface of said implant is treated to partially expose inner composition.

Optionally the surface maximum roughness is more than 2 microns.

Optionally the surface maximum roughness is more than 3 microns.

Optionally said body composition comprises more than 8% w/w silica but said surface composition comprises less than 4% w/w silica.

Optionally the implant comprises a plurality of holes. Optionally said holes comprise an inner surface that is different from said surface of the implant. Optionally said inner surface comprises a different composition. Optionally said holes comprise an inner surface comprising a composition of said surface of the implant.

According to at least some embodiments, there is provided a method of precise ablation of polymer material from a surface of an implant, and implants with such an ablated surface. The depth to which this occurs is preferably controlled. Also preferably the structure of fiber is preserved, such that only the surface polymer is removed. There is preferably a low amount of depth variation and square area variation.

Ablation can optionally be achieved through any suitable method, including but not limited to an erosive method, including mechanical brushing, cutting or chipping, and/or irradiation or laser ablation. Preferably ablation is achieved through irradiation or laser ablation.

The polymer material is ablated from the surface of the implant without damaging the mineral fibers and without compressing them so they retain their structure intact and are not frayed. In terms of fiber diameter, preferably the fiber diameter ranges in a range of 2-40 microns, and more preferably 4-20 microns fiber diameter.

Preferably the polymer surface is ablated to a controlled extent, such that a structure of said fibers is maintained open ablation of the polymer surface. Also preferably the fiber structure is maintained, wherein at least 50, 65, 80, 85, 90, 95% of surface fibers retain their geometric structure or are not ablated or do not have a portion of the fiber removed. For example, optionally the depth of the polymer surface is in the range of 1-100 micron, 5-50 micron, or 10-30 micron. Optionally the fiber diameter is in a range of 2-40 microns, and more preferably 4-20 microns fiber diameter.

In various embodiments, different amounts of the outer surface of the medical implant may be surface treated with ablation. For example, 10-70% of the medical implant outer surface may be surface treated with ablation. Optionally, 30-55% of the medical implant outer surface is surface treated. Also optionally, 15-40% of the medical implant outer surface is surface treated.

Various embodiments may feature different depths of ablation, as measured from the outer surface of the implant. For example, a depth of the ablation optionally ranges from 1 to 120 microns from outer surface. Optionally the depth of the ablation ranges from 5 to 70 microns. Also optionally, the depth of the ablation ranges from 5 to 40 microns.

In various exemplary implementations of the implant, the fibers are arranged in layers. Optionally, the depth of the ablation ranges from 1 to 50 microns into the top layer of the fibers. Preferably, the depth of the ablation ranges from 3 to 20 microns. Ablation depth may be considered with regard to implant wall thickness and/or overall implant thickness. For example, the ablation depth may be in a range of 0.1% to 10% of implant wall thickness or implant overall thickness. Optionally, the depth of the ablation ranges from 0.5% to 2.5%.

Optionally a shape of an area of treatment of the outer surface is selected from the group consisting of rectangular, square, circular, arc shaped, diamond, parallelograms, triangular or any combination of said shapes.

Optionally a shape of an area of treatment of the outer surface comprises a line shape of specified width, wherein said surface treated line width is up to 100 microns. For example, optionally the line shape comprises one or more of continuous solid line, dashed line, dotted line, circumferential line, angled line (any angle from 5 to 85 degrees), helix line (helix angle of 5 to 85 degrees). Preferably the width is in a range of from 5 microns to 100. Optionally the surface treated line width is in a range of from 10 microns to 70 microns. Preferably the surface treated line width is in a range of from 20 microns to 40 microns.

According to various embodiments, the fibers may have different orientations in the implant. For example, optionally the surface treatment exposes fibers of different orientations on the outer surface. Optionally the exposed fiber orientation is parallel to the medical implant body axis. Also optionally, the exposed fiber orientation is 5°-85° relative to the implant body axis. Preferably, the exposed fiber orientation is 159-65° relative to the implant body axis. More preferably, the exposed fiber orientation is 30°-60° relative to the implant body axis.

Optionally, the exposed fibers have more than one direction on the treated surface. These different directions may be realized, for example, according to an angle between neighboring areas having different fiber directions. Optionally, the angle between one area of fiber direction to its neighboring area with different fiber direction is between 0°-90°. Also optionally, the angle between one area of fiber direction to its neighboring area with different fiber direction is between 25°-75° Optionally, the cross-sectional fiber exposure is 90° relative to the fiber axis. Optionally, the cross-sectional fiber exposure is 15°-65° relative to the fiber axis. Optionally, the cross-sectional fiber exposure comprises more than one fiber direction.

Surface maximum roughness may also be controlled through ablation. Controlling surface maximum roughness may, without wishing to be limited by a closed list, lead to such benefits as better ingrowth of tissue, better adherence to tissue and so forth. Optionally surface maximum roughness is more than 1-10 microns. Preferably the surface maximum roughness is more than 3-8 microns. Also preferably, the surface maximum roughness is more than 4-6 microns.

After ablation, various resultant surface geometries may occur in regard to the exposed fibers. For example, the surface may be exposed fibers alone. The exposed fibers may comprise 20-80% of the ablated surface. Optionally, the exposed fibers comprise 35-65% of the ablated surface. Optionally, the exposed fibers comprise 51-70% of the ablated surface.

After ablation, various surface geometries may also be provided with different shapes. For example, optionally after ablation the resultant surface geometry is step shaped. Optionally the implant may comprise a plurality of ribs or threads, wherein said polymer surface is thicker on the implant body comparing to said polymer surface thickness on the ribs/threads. For example, the ribs/threads may be treated while a remainder of the implant is untreated, or vice versa.

In one embodiment, outermost surface region has been modified to increase roughness and/or porosity.

Optionally roughness is defined by presence of promontories, prominences or protuberances on the surface of the implant with height equal to or less than the depth of the outermost surface region. Preferably such promontories, prominences or protuberances are less than 5 microns in diameter, on average. More preferably, less than 3, less than 2, less than 1 micron in average diameter. Optionally such promontories, prominences or protuberances are present in the outermost surface area but absent in the innermost surface area.

Optionally roughness is defined by Ra measure in nanometers (nm). Preferably roughness in modified outermost surface area is greater than 100 nm, more preferably greater than 200 nm, and most preferably greater than 300 nm. Preferably roughness in unmodified surface area is less than 100 nm.

Optionally, porosity is defined as full thickness pore (holes) in the entire surface region or outermost surface layer. Preferably, implant is a mineral fiber-reinforced implant and porosity in surface layer exposes mineral fibers.

According to at least some embodiments, there is provided a biocomposite medical implant with a modified surface wherein the outermost surface layer of the implant is comprised of a majority of bioabsorbable polymer but wherein the surface has been modified such that the surface of the implant comprises roughness, texture, or porosity such that an increased amount of mineral composition is exposed as compared with the outermost surface layer of the implant.

Outermost surface layer as used herein may define the outermost 1-100 μm of the implant. Preferably the outermost 1-20 μm of the implant, more preferable the outermost 1-10, and most preferably the outer 1-5.

The exposed mineral composition may comprise the mineral composition that is part of the biocomposite composition. The mineral composition may optionally or additionally comprise another mineral such as Hydroxyapatite, Calcium Phosphate, Calcium Sulfate, Dicalcium Phosphate, Tricalcium Phosphate.

The roughness or texture of the surface may include exposure of the internal composition of the implant to a depth of the outermost 1-100 μm of the implant. Preferably the outermost 1-20 μm of the implant, more preferable the outermost 1-10, and most preferably the outer 1-5 microns.

Preferably, the outermost layer of the implant comprises at least 30% polymer, more preferably at least 50%, more preferably at least 70%, and most preferably at least 80%.

The composition of the biocomposite is comprised of at least 20% mineral composition, preferably at least 30%, more preferably at least 40%, and most preferably at least 50%.

Preferably the composition of the outermost layer of the implant comprises a greater percentage of polymer than the overall composition of the implant. Preferably, at least 10% more, 20%, 30%, 50%

Optionally, the modified surface of the implant includes pores in the polymer surface. The average pore diameter is preferably in the range of 1-500 μm, more preferably in the range 10-300 μm, more preferably in the range 50-250 μm.

Preferably, surface is modified with surface treatment using grit blasting.

Preferably grit is comprised of a biocompatible material.

Preferably grit is comprised of a combination of Hydroxyapatite, Calcium Phosphate, Calcium Sulfate, Dicalcium Phosphate, and Tricalcium Phosphate.

Preferably grit is of an average diameter size in the range of 10-500 μm. More preferably in the range of 20-120 μm.

### Bioabsorbable Polymers

In a preferred embodiment of the present invention, the biodegradable composite comprises a bioabsorbable polymer.

The medical implant described herein may be made from any biodegradable polymer. The biodegradable polymer may be a homopolymer or a copolymer, including random copolymer, block copolymer, or graft copolymer. The biodegradable polymer may be a linear polymer, a branched polymer, or a dendrimer. The biodegradable polymers may be of natural or synthetic origin. Examples of suitable biodegradable polymers include, but are not limited to polymers such as those made from lactide, glycolide, caprolactone, valerolactone, carbonates (e.g., trimethylene carbonate, tetramethylene carbonate, and the like), dioxanones (e.g., 1,4-dioxanone), 6-valerolactone, 1, dioxepanones) e.g., 1,4-dioxepan-2-one and 1,5-dioxepan-2-one), ethylene glycol, ethylene oxide, esteramides, γ-ydroxyvalerate, β-hydroxypropionate, alpha-hydroxy acid, hydroxybuterates, poly (ortho esters), hydroxy alkanoates, tyrosine carbonates, polyimide carbonates, polyimino carbonates such as poly (bisphenol A-iminocarbonate) and poly (hydroquinone-iminocarbonate, (polyurethanes, polyanhydrides, polymer drugs (e.g., polydiflunisol, polyaspirin, and protein therapeutics (and copolymers and combinations thereof. Suitable natural biodegradable polymers include those made from collagen, chitin, chitosan, cellulose, poly (amino acids), polysaccharides, hyaluronic acid, gut, copolymers and derivatives and combinations thereof.

According to the present invention, the biodegradable polymer may be a copolymer or terpolymer, for example: polylactides (PLA), poly-L-lactide (PLLA), poly-DL-lactide (PDLLA); polyglycolide (PGA); copolymers of glycolide, glycolide/trimethylene carbonate copolymers (PGA/TMC); other copolymers of PLA, such as lactide/tetramethylglycolide copolymers, lactide/trimethylene carbonate copolymers, lactide/d-valerolactone copolymers, lactide/ε-caprolactone copolymers, L-lactide/DL-lactide copolymers, glycolide/L-lactide copolymers (PGA/PLLA), polylactide-co-glycolide; terpolymers of PLA, such as lactide/glycolide/trimethylene carbonate terpolymers, lactide/glycolide/ε-caprolactone terpolymers, PLA/polyethylene oxide copolymers; polydepsipeptides; unsymmetrically-3,6-substituted poly-1,4-dioxane-2,5-diones; polyhydroxyalkanoates; such as polyhydroxybutyrates (PHB); PHB/b-hydroxyvalerate copolymers (PHB/PHV); poly-b-hydroxypropionate (PHPA); poly-p-dioxanone (PDS); poly-d-valerolactone-poly-ε-capralactone, poly(ε-caprolactone-DL-lactide) copolymers; methylmethacrylate-N-vinyl pyrrolidone copolymers; polyesteramides; polyesters of oxalic acid; polydihydropyrans; polyalkyl-2-cyanoacrylates; polyurethanes (PU); polyvinylalcohol (PVA); polypeptides; poly-b-malic acid (PMLA): poly-b-alkanbic acids; polycarbonates; polyorthoesters; polyphosphates; poly(ester anhydrides); and mixtures thereof; and natural polymers, such as sugars; starch, cellulose and cellulose derivatives, polysaccharides, collagen, chitosan, fibrin, hyalyronic acid, polypeptides and proteins. Mixtures of any of the above-mentioned polymers and their various forms may also be used.

The biodegradable composite is preferably embodied in a polymer matrix, which may optionally comprise any of the above polymers. Optionally and preferably, it may comprise a polymer selected from the group consisting of a bioabsorbable polyester, PLLA (poly-L-lactide), PDLLA (poly-DL-lactide), PLDLA, PGA (poly-glycolic acid), PLGA (poly-lactide-glycolic acid), PCL (Polycaprolactone), PLLA-PCL and a combination thereof. If PLLA is used, the matrix preferably comprises at least 30% PLLA, more preferably 50%, and most preferably at least 70% PLLA. If PDLA is used, the matrix preferably comprises at least 5% PDLA, more preferably at least 10%, most preferably at least 20% PDLA.

Optionally, the inherent viscosity (IV) of the polymer matrix (independent of the reinforcement fiber) is in the range of 0.2-6 dl/g, preferably 1.0 to 3.0 dl/g, more preferably in the range of 1.5 to 2.4 dl/g, and most preferably in the range of 1.6 to 2.0 dl/g.

Inherent Viscosity (IV) is a viscometric method for measuring molecular size. IV is based on the flow time of a polymer solution through a narrow capillary relative to the flow time of the pure solvent through the capillary.

### Reinforced Biocomposite

According to at least some embodiments of the present invention, the medical implant comprises a reinforced biocomposite (i.e. a bioabsorbable composite that includes the previously described polymer and also incorporates a reinforcing filler, generally in fiber form, to increase the mechanical strength of the polymer). For the avoidance of doubt, the terms “filler” and “fiber” are used interchangeably to describe the reinforcing material structure.

In a more preferred embodiment of the present invention, the reinforced bioabsorbable polymer is a reinforced polymer composition comprised of any of the above-mentioned bioabsorbable polymers and a reinforcing filler, preferably in fiber form. The reinforcing filler may be comprised of organic or inorganic (that is, natural or synthetic) material. Reinforcing filler may be a biodegradable glass or glass-like materials, a ceramic, a mineral composition (optionally including one or more of hydroxyapatite, tricalcium phosphate, calcium sulfate, calcium phosphate), a cellulosic material, a nano-diamond, or any other filler known in the art to increase the mechanical properties of a bioabsorbable polymer. The filler may also optionally be a fiber of a bioabsorbable polymer itself. Preferably, reinforcing fiber is comprised of a bioabsorbable glass, ceramic, or mineral composition.

Preferably, reinforcement fiber is comprised of silica-based mineral compound such that reinforcement fiber comprises a bioresorbable glass fiber, which can also be termed a bioglass fiber composite.

According to at least some embodiments, bioresorbable glass fiber may optionally have oxide compositions in the following mol. % ranges (as a percent over the glass fiber composition);
NO: 11.0-19.0 mol. %
CaO: 9.0-14.0 mol %
MgO: 1.5-8.0 mol. %
B₂O₃: 0.5-3.0 mol %
Al₂O₃: 0-0.8 mol. %
P₂O₃: 0.1-0.8 mol. %
SiO₂: 67-73 mol. %
but preferably in the following mol. % ranges:
Na₂O: 12.0-13.0 mol. %
CaO: 9.0-10.0 mol. %
MgO: 7.0-8.0 mol. %
B₂O₃: 1.4-2.0 mol. %
P₂O₃: 0.5-0.8 mol. %
SiO₂: 68-70 mol. %

Additional optional bioresorbable glass compositions are described in the following patent applications, which are hereby incorporated by reference as if fully set forth herein: Biocompatible composite and its use (WO2010122098); and Resorbable and biocompatible fiber glass compositions and their uses (WO2010122019). Tensile strength of the reinforcement fiber is preferably in the range of 1200-2800 MPa, more preferably in the range of 1600-2400 MPa, and most preferably in the range of 1800-2200 MPa.

Elastic modulus of the reinforcement fiber is preferably in the range of 30-100 GPa, more preferably in the range of 50-80 GPa, and most preferably in the range of 60-70 GPa.

Reinforcing filler is preferably incorporated in the bioabsorbable polymer matrix of the biocomposite in fiber form. Preferably, such fibers are continuous fibers.

Preferably continuous fibers are aligned within the implant such that the ends of fibers do not open at the surface of the implant.

Preferably, fibers are distributed evenly within the implant.

Specifically within bioabsorbable fiber-reinforced composites, achieving the high strengths and stiffness required for many medical implant applications can require the use of continuous-fiber reinforcement rather than short or long fiber reinforcement. This creates a significant difference from the implant structures, architectures, designs, and production techniques that have been previously used with medical implants produced from polymers or composites comprising short or long fiber reinforced polymers. Those implants are most commonly produced using injection molding, or occasionally 3-D printing, production techniques. The production of these implants generally involves homogeneity of the material throughout the implant and the finished implant is then comprised of predominantly isotropic material. However, with continuous fiber-reinforcement, the fibers must be carefully aligned such that each fiber or bundle of fibers runs along a path within the composite material such that they will provide reinforcement along specific axes within the implant to provide stress resistance where it is most needed.

The present invention provides, in at least some embodiments, implant compositions from continuous-fiber reinforced bioabsorbable composite materials that are a significant step forward from previous bioabsorbable implants in that they can achieve sustainably high, load bearing strengths and stiffness. Additionally, many embodiments of the present invention additionally facilitate these high strength levels with efficient implants of low volume since the anisotropic nature of the implants can allow the implants to achieve high mechanical properties in axes where those properties are needed (for example in bending resistance) without necessitating the additional volume that would be needed to uniformly provide high mechanical properties in all other axes.

According to at least some embodiments, there is provided a medical implant comprising a plurality of composite layers, said layers comprising a biodegradable polymer and a plurality of uni-directionally aligned continuous reinforcement fibers. Optionally and preferably, the biodegradable polymer is embodied in a biodegradable composite. Also optionally and preferably, the fibers are embedded in a polymer matrix comprising one or more bioabsorbable polymers.

According to at least some embodiments, the composite layers are each comprised of one or more composite tapes, said tape comprising a biodegradable polymer and a plurality of uni-directionally aligned continuous reinforcement fibers. Optionally and preferably, the biodegradable polymer is embodied in a biodegradable composite. Also optionally and preferably, the fibers are embedded in a polymer matrix comprising one or more bioabsorbable polymers.

Preferably, the composite tape layer comprises reinforcement fibers that are pre-impregnated with polymer.

Preferably, each composite layer is of thickness 0.05 mm-0.5 mm, more preferably 0.15-0.35 mm, and most preferably 0.1-0.25 mm.

Preferably, each composite tape is of width 2-30 mm, more preferably tape is of width 4-16 mm, and most preferably of width 6-12 mm.

Preferably, reinforcement fiber content within the composite tape is in the range of 20-70%, more preferably in the range of 30-60%, more preferably in the range of 40-50%, and most preferably 45-50% over the entire composite tape materials. Optionally and preferably, the fiber-reinforced biodegradable composite within the implant has a flexural modulus exceeding 10 GPa and flexural strength exceeding 100 MPa.

Optionally, the fiber-reinforced biodegradable composite within the implant has flexural strength in range of 200-1000 MPa, preferably 300-800 MPa, more preferably in the range of 400-800 MPa, and most preferably in the range of 500-800 MPa

Optionally, the fiber-reinforced biodegradable composite within the implant has elastic modulus in range of 10-30 GPa, preferably 12-28 GPa, more preferably in the range of 16-28 GPa, and most preferably in the range of 20-26 GPa.

Optionally, fibers may be aligned at an angle to the longitudinal axis (i.e. on a diagonal) such that the length of the fiber may be greater than 100% of the length of the implant. Optionally and preferably, a majority of reinforcement fibers are aligned at an angle that is less than 90°, alternatively less than 60°, or optionally less than 45° from the longitudinal axis.

Preferably, the implant preferably comprises between 2-20 composite tape layers, more preferably between 2-10 layers, and most preferably between 2-6 layers; wherein each layer may be aligned in a different direction or some of the layers may be aligned in the same direction as the other layers.

Preferably, the maximum angle between fibers in at least some of the layers is greater than the angle between the fibers in each layer and the longitudinal axis. For example, one layer of reinforcing fibers may be aligned and a right diagonal to the longitudinal axis while another layer may be aligned at a left diagonal to the longitudinal axis.

Optionally and preferably, the composite composition additionally includes a compatibilizer, which for example be such an agent as described in WO2010122098, hereby incorporated by reference as if fully set forth herein.

Reinforcing fiber diameter preferably in range of 2-40 um, preferably 8-20 um, most preferably 12-18 um (microns).

Preferably, the implant includes only one composition of reinforcing fiber.

Preferably fibers don't open at the surface of the implant.

Numerous examples of reinforced polymer compositions have previously been documented. For example: A biocompatible and resorbable melt derived glass composition where glass fibers can be embedded in a continuous polymer matrix (EP 2 243 749 A1), Biodegradable composite comprising a biodegradable polymer and 20-70 vol % glass fibers (WO2010128039 A1), Resorbable and biocompatible fiber glass that can be embedded in polymer matrix (US 2012/0040002 A1), Biocompatible composite and its use (US 2012/0040015 A1), Absorbable polymer containing poly [succinimide] as a filler (EP0 671 177 B₁).

In a more preferred embodiment of the present invention, the reinforcing filler is covalently bound to the bioabsorbable polymer such that the reinforcing effect is maintained for an extended period. Such an approach has been described in US 2012/0040002 A1 and EP 2243500B₁, hereby incorporated by reference as if fully forth herein, which discusses a composite material comprising biocompatible glass, a biocompatible matrix polymer and a coupling agent capable of forming covalent bonds.

### Fabrication of the Implant

Any of the above-described bioabsorbable polymers or reinforced bioabsorbable polymers may be fabricated into any desired physical form for use with the present invention. The polymeric substrate may be fabricated for example, by compression molding, casting, injection molding, pultrusion, extrusion, filament winding, composite flow molding (CFM), machining, or any other fabrication technique known to those skilled in the art. The polymer may be made into any shape, such as, for example, a plate, screw, nail, fiber, sheet, rod, staple, clip, needle, tube, foam, or any other configuration suitable for a medical device.

### Load-Bearing Mechanical Strength

The present invention particularly relates to bioabsorbable composite materials that can be used in medical applications that require high strength and a stiffness compared to the stiffness of bone. These medical applications require the medical implant to bear all or part of the load applied by or to the body and can therefore be referred to generally as “load-bearing” applications. These include bone fixation, fracture fixation, tendon reattachment, joint replacement, spinal fixation, and spinal cages.

The flexural strength preferred from a bioabsorbable composite (such as a reinforced bioabsorbable polymer) for use in the load-bearing medical implant is at least 200 MPa, preferably above 400 MPa, more preferably above 600 MPa, and even more preferably above 800 MPa. The Elastic Modulus (or Young's Modulus) of the bioabsorbable composite for use with present invention is preferably at least 10 GPa, more preferably above 15 GPa, and even more preferably above 20 GPa but not exceeding 100 GPa and preferably not exceeding 60 GPa.

### Sustained Mechanical Strength

There is a need for the bioabsorbable load-bearing medical implants of the present invention to maintain their mechanical properties (high strength and stiffness) for an extended period to allow for sufficient bone healing. The strength and stiffness preferably remains above the strength and stiffness of cortical bone, approximately 150-250 MPa and 15-25 GPa respectively, for a period of at least 3 months, preferably at least 6 months, and even more preferably for at least 9 months in vivo (i.e. in a physiological environment).

More preferably, the flexural strength remains above 400 MPa and even more preferably remains above 600 MPa.

The present invention overcomes the limitations of previous approaches and provides medical implants comprised of biodegradable compositions that retain their high mechanical strength and stiffness for an extended period sufficient to fully support bone regeneration d rehabilitation

### Material Specific Design Benefits

Without wishing to be limited by a closed list, the material-specific design benefits are optionally provided by one or more of the following unique characteristics of implants manufactured from this material:

1. Absorbable structural implants wherein strength and stiffness properties are anisotropic. The bending resistance and other mechanical properties of these implants depends greatly on the specific design of the part and of the alignment of reinforcing fibers within the part. It is therefore possible to design such implants efficiently such that they provide sufficient support in the necessary axes (for example, flexural stiffness) without comprising an excessive amount of material that would provide equivalent support in the remaining axes (for example, tensile stiffness).

2. Low profile/minimally invasive/material efficient design for absorbable implant that take advantage of the strength and stiffness characteristics of the reinforced absorbable composite material to create implants that achieve bone fixation with minimal profile. By “minimal profile”, it is meant that the implant is reduced in size in at least one dimension in comparison with an equivalent currently available implant that is not made from such composite material.

3. Load bearing absorbable bone implants, as opposed to previous absorbable implants which did not approach the stiffness of cortical bone.

4. Small functional features, such as anchors, ridges, teeth, etc that require the reinforcement in order to be strong enough to be functional. Previous absorbable materials may not have had sufficient strength for such features.

5. The capability of being produced according to fiber-reinforced composite specific manufacturing techniques such as compression molding, pultrusion, etc.

6. Reduced damage to surrounding tissues, including both soft tissues and bone tissues, as compared with the trauma of stress risers or stress shielding that can arise from use of high modulus (such as metal) implants.

The present invention, according to at least some embodiments, thus provides medical implants that are useful as structural fixation for load-bearing purposes, exhibiting sustained mechanical properties.

The present invention, according to at least some embodiments, further comprises a biodegradable composite material in which the drawbacks of the prior art materials can be minimized or even eliminated, i.e. the composite retains its strength and modulus in vivo for a time period sufficient for bone healing for example. Mechanical strength as used here includes, but is not limited to, bending strength, torsion strength, impact strength, compressive strength and tensile strength.

The presently claimed invention, in at least some embodiments, relate to a biocomposite material comprising a biocompatible polymer and a plurality of reinforcing fibers, wherein said reinforcing fibers are oriented in a parallel orientation.

The biocomposite material has one or more mechanical properties which feature an increased extent or degree as compared to such a material with reinforcing fibers oriented in a non-parallel orientation. Optionally such a non-parallel orientation is a perpendicular or amorphous (non-oriented) orientation. elastic modulus, tensile modulus, compression modulus, shear modulus, bending moment, moment of inertia, bending strength, torsion strength, shear strength, impact strength, compressive strength and/or tensile strength. The increased extent or degree may optionally be at least twice as great, at least five times as great, at least ten times as great, at least twenty times as great, at least fifty times as great, or at least a hundred times as much, or any integral value in between.

Optionally the mechanical properties can comprise any one of Flexural strength, Elastic modulus and Maximum load, any pair of same or all of them. Optionally density and/or volume are unchanged or are similar within 5%, within 10%, within 15%, within 20%, any integral value in between or any integral value up to 50%.

Optionally the biocomposite implant as described herein is swellable, having at least 0.5% swellability, at least 1%, 2% swellability, and less than 20% swellability, preferably less than 10% or any integral value in between.

Optionally, the swellability in one mechanical axis is greater than the swellability in a second mechanical axis. Preferably the difference in swelling percentage (%) between axes is at least 10%, at least 25%, at least 50%, or at least 100%, or any integral value in between.

After exposure to biological conditions for 1 hour, 12 hours, 24 hours, 48 hours, five days, one week, one month, two months of six months or any time value in between, the biocomposite material implants preferably retain at least 10%, at least 20%, at least 50%, at least 60%, at least 75%, at least 85% or up to 100% of flexural strength, Modulus and/or Max load, and/or volume, or any integral value in between. By “biological conditions” it is meant that the temperature is between 30-40° C. but preferably is at 37° C. Optionally, fluid conditions replicate those in the body as well, under “simulated body fluid” conditions.

The flexural strength of the implant or segment of the implant is preferably at least 200 MPA, at least 400 mPa, at least 600 mPA, at least 1000 mPA or any integral value in between.

Relevant implants may include bone fixation plates, intramedullary nails, joint (hip, knee, elbow) implants, spine implants, and other devices for such applications such as for fracture fixation, tendon reattachment, spinal fixation, and spinal cages.

According to at least some embodiments, there are provided medical implants for bone or soft tissue fixation comprising a biodegradable composite, wherein said composite optionally and preferably has the following properties:
(i) wherein biodegradable composite comprises one or more biodegradable polymers and a resorbable, reinforcement fiber; and
(ii) wherein one or more segments comprising the medical implant have a maximum flexural modulus in the range of 6 GPa to 30 GPa and flexural strength in the range of 100 MPa to 1000 MPa; and
(iii) wherein the average density of the composite is in the range of 1.1-3.0 g/cm³.

Preferably, average density of the composite is in the range of 1.2-2.0 g/cm³.

More preferably, average density of the composite is in the range of 1.3-1.6 g/cm³.

Preferably, flexural modulus is in the range of 10 GPa to 28 GPa and more preferably in the range of 15 to 25 GPa.

Preferably, flexural strength is in the range of 200-800 MPa. More preferably, 400-800 MPa.

In a preferred embodiment of the present invention, at least 50% of elastic modulus is retained following exposure to simulated body fluid (SBF) at 50° C. for 3 days. More preferably at least 70% is retained, and even more preferably at least 80% is retained.

In a preferred embodiment of the present invention, at least 20% of strength is retained following exposure to simulated body fluid (SBF) at 50° C. for 3 days. More preferably at least 30% is retained, and even more preferably at least 40% is retained.

In a preferred embodiment of the present invention, at least 50% of elastic modulus is retained following exposure to simulated body fluid (SBF) at 37° C. for 3 days. More preferably at least 70%, and even more preferably at least 85%.

In a preferred embodiment of the present invention, at least 30% of strength is retained following exposure to simulated body fluid (SBF) at 37° C. for 3 days. More preferably at least 45%, and even more preferably at least 60%.

Specifically regarding medical implants described herein that contain one or more segments that can be anisotropic, this anisotropicity reflects a significant divergence from what has be previously accepted in medical, and specifically orthopedic, implants in that the anisotropic structure results in implants in which there are mechanical properties in one or more axis that are less than the optimal mechanical properties which may be achieved by the materials from which the implant is comprised. In contrast, traditional implants have relied upon the uniform mechanical properties of the materials from which they are comprised as this does not require compromising in any axis.

The anisotropic approach can only be applied following biomechanical analysis to determine that greater implant mechanical properties is required in certain axes as opposed to other axes. For example, an implant may be subjected to very high bending forces but only nominal tensile forces and therefore require a much greater emphasis on bending forces. Other relevant axes of force in a medical implant can include tensile, compression, bending, torsion, shear, pull-out (from bone) force, etc.

There are several factors that affect the mechanical properties of an implant. As described above, material composition alone results in a generally uniform of isotropic structure. Without wishing to be limited by a closed list or a single hypothesis, within fiber-reinforced biocomposite medical implants, an anisotropic structure may result from one or more of the following characteristics:
1. The weight ratio of reinforcing fibers to biopolymer. Preferably this ratio is in the range of 1:1 to 3:1 and more preferably 1.5:1 to 2.5:1.
2. The density of the medical implant (this characteristic is also determined to some extent the ratio of reinforcing fiber to polymer)
3. The diameter of reinforcing fiber. The average fiber diameter is preferably between 5 and 50 μm. More preferably between 10-30 μm.
4. Length of fiber (continuous fiber, long fiber, short fiber). Preferably, having continuous fiber reinforcement with fibers that run across the entire implant.
5. The alignment of fibers or fiber layers. Preferably, in each segment of the implant, a majority of fibers or fiber layers are aligned or partially aligned with the axis that will be exposed to the highest bending forces. If partially aligned, then preferably within a 45° angle of the axis.
6. The number of fibers or fiber layers aligned in any given direction. Preferably fiber layers are 0.1 to 1 mm in thickness and more preferably 0.15 to 0.25 mm.
7. The order of fiber layers.

In one embodiment of the present invention, the medical implant is a pin, screw, or wire.

Preferably, a pin or wire of 2 mm external diameter will have a shear load carrying capacity of greater than 200 N. More preferably shear load carrying capacity of 2 mm pin will exceed 400 N and most preferably will exceed 600 N.

### Clinical Applications

The medical implants discussed herein are generally used for bone fracture reduction and fixation to restore anatomical relationships. Such fixation optionally and preferably includes one or more, and more preferably all, of stable fixation, preservation of blood supply to the bone and surrounding soft tissue, and early, active mobilization of the part and patient.

There are several exemplary, illustrative, non-limiting types of bone fixation implants for which the materials and concepts described according to at least some embodiments of the present invention may be relevant, as follows:

### Screws

Screws are used for internal bone fixation and there are different designs based on the type of fracture and how the screw will be used. Screws come in different sizes for use with bones of different sizes. Screws can be used alone to hold a fracture, as well as with plates, rods, or nails. After the bone heals, screws may be either left in place or removed.

Screws are threaded, though threading can be either complete or partial. Screws can include compression screws, locking screws, and/or cannulated screws. External screw diameter can be as small as 0.5 or 1.0 mm but is generally less than 3.0 mm for smaller bone fixation. Larger bone cortical screws can be up to 5.0 mm and cancellous screws can even reach 7-8 mm. Some screws are self-tapping and others require drilling prior to insertion of the screw. For cannulated screws, a hollow section in the middle is generally larger than 1 mm diameter in order to accommodate guide wires.

### Wires/Pins

Wires are often used to pin bones back together. They are often used to hold together pieces of bone that are too small to be fixed with screws. They can be used in conjunction with other forms of internal fixation, but they can be used alone to treat fractures of small bones, such as those found in the band or foot. Wires or pins may have sharp points on either one side or both sides for insertion or drilling into the bone.

“K-wire” is a particular type of wire generally made from stainless steel, titanium, or nitinol and of dimensions in the range of 0.5-2.0 mm diameter and 2-25 cm length. “Steinman pins” are general in the range of 2.0-5.0 mm diameter and 2-25 cm length. Nonetheless, the terms pin and wire for bone fixation are used herein interchangeably.

### Anchors

Anchors and particularly suture anchors are fixation devices for fixing tendons and ligaments to bone. They are comprised of an anchor mechanism, which is inserted into the bone, and one or more eyelets, holes or loops in the anchor through which the suture passes. This links the anchor to the suture. The anchor which is inserted into the bone may be a screw mechanism or an interference mechanism. Anchors are generally in the range of 1.0-6.5 mm diameter.

### Cable, Ties, Wire Ties

Cables, ties, or wire ties (one example of wire tie is Synthes ZipFix™) can be used to perform fixation by cerclage, or binding, bones together. Such implants may optionally hold together bone that cannot be fixated using penetration screws or wires/pin, either due to bone damage or presence of implant shaft within bone. Generally, diameter of such cable or tie implants is optionally in the range of 1.0 mm-2.0 mm and preferably in the range of 1.25-1.75 mm. Wire tie width may optionally be in the range of 1-10 mm.

### Nails or Rods

In some fractures of the long bones, medical best practice to hold the bone pieces together is through insertion of a rod or nail through the hollow center of the bone that normally contains some marrow. Screws at each end of the rod are used to keep the fracture from shortening or rotating, and also hold the rod in place until the fracture has healed. Rods and screws may be left in the bone after healing is complete. Nails or rods for bone fixation are generally 20-50 cm in length and 5-20 mm in diameter (preferably 9-16 mm). A hollow section in the middle of nail or rod is generally larger than 1 mm diameter in order to accommodate guide wires.

Other non-limiting, illustrative examples of bone fixation implants may optionally include plates, plate and screw systems, and external fixators.

Any of the above-described bone fixation implants may optionally be used to fixate various fracture types including but not limited to comminuted fractures, segmental fractures, non-union fractures, fractures with bone loss, proximal and distal fractures, diaphyseal fractures, osteotomy sites, etc.

### EXAMPLES

### Example 1-Mineral Content of Ribs/Threads and Core in Ribbed/Threaded Implants

Below example describes production of ribbed and threaded implants with reinforced biocomposite materials. This example demonstrates how different medical implant pins comprised of reinforced biocomposite materials have different mineral composition in their bodies and in their ribs or threads.

### Materials & Methods

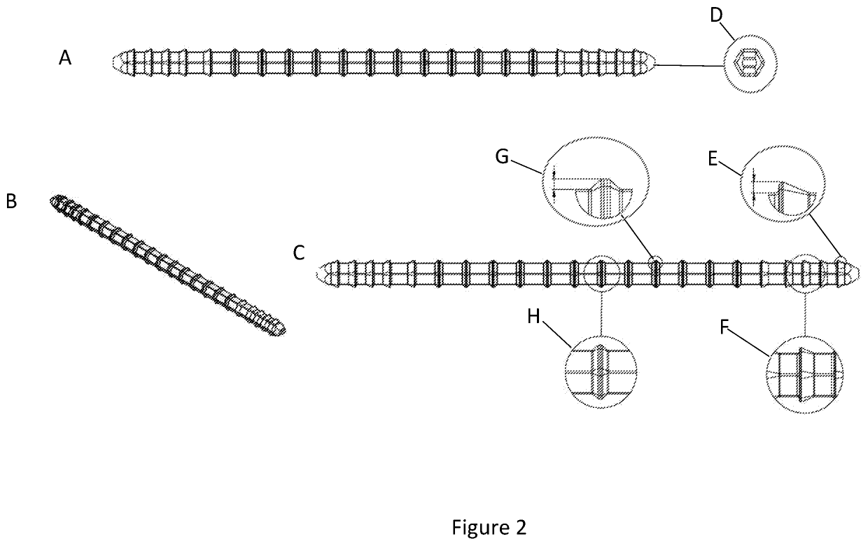

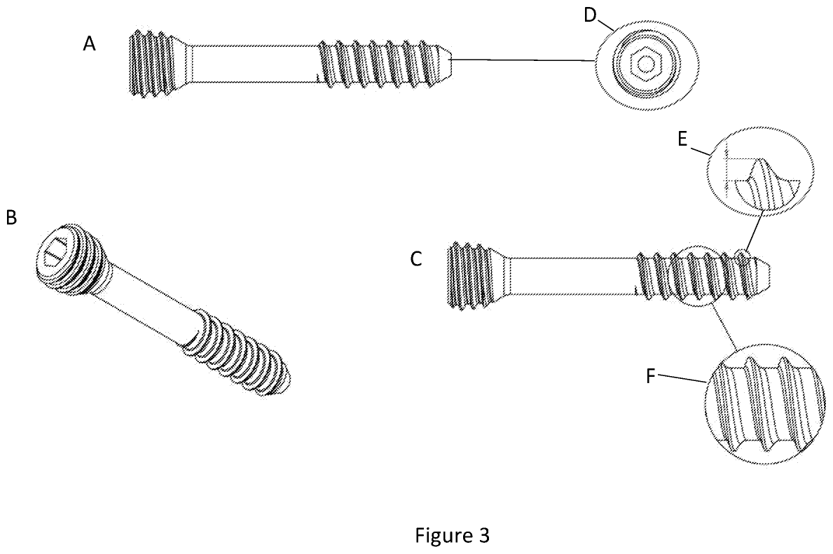

Three types of implants were produced using reinforced composite material.
1. 4.0 mm×50 mm ribbed hexagonal nail (FIG. 1): Edge to edge core diameter of 3.4 mm and edge to edge rib diameter of 3.9 mm. (i.e. rib height of 0.25 mm).
2. 2.4 mm×50 mm ribbed hexagonal nail (FIG. 2): Edge to edge core diameter of 2.0 mm and edge to edge rib diameter of 2.5 mm. (i.e. rib height of 0.25 mm).
3. 4.0 mm×50 mm compression screw (FIG. 3): Core diameter of 3.0 mm and thread edge to thread edge diameter of 4.0 mm. (i.e. thread height of 0.5 mm).

Three samples were prepared for each type of implant.

Material composite raw material was comprised of PLDLA 70/30 polymer reinforced with 50% w/w continuous mineral fibers. Mineral fibers composition was approximately Na₂O 14%, MgO 5.4%, CaO) 9%, B₂O₃: 2.3%, P₂O₅ 1.5%, and SiO₂ 67.8% w/w. Testing samples were manufactured by compression molding of multiple layers of composite material into a mold.

For each implant, the ribs or threads were cut and detached from the core of the implant using a scalpel. The ribs removed from each implant were weighed and the remaining implant core was weighed.

The mineral content of the ribs and the core for each implant sample were measured using residue on ignition method. Each of the ribs and the core were placed in a crucible and placed in a muffle furnace heated to 600° C. for 3 hours until all the carbonaceous material has disappeared. The crucibles were then removed from the muffle furnace, placed into the desiccator, and let to cool down to ambient temperature for at least 1 hour. The samples were then weighed with the crucibles using analytical balance.

The weight measured following the residue on ignition method was determined to be the mineral fiber content of that part of the sample (ribs or core).

### Results

1. 4.0 mm×50 mm hexagonal nail

### Sample 1

| | | | | crucible + | % |
|---|---|---|---|---|---|
| | empty | crucible + | sample | sample after | mineral |
| Sample | crucible | sample | weight | ignition | fiber |
| Core | 22.5289 | 25.0305 | 2.5016 | 23.7965 | 50.7 |
| Ribs | 23.5951 | 23.6167 | 0.0216 | 23.5989 | 17.6 |

### Sample 2

| | | | | crucible + | % |
|---|---|---|---|---|---|
| | empty | crucible + | sample | sample after | mineral |
| Sample | crucible | sample | weight | ignition | fiber |
| Core | 22.8404 | 26.1697 | 3.3293 | 24.4824 | 49.32 |
| Ribs | 23.1967 | 23.2658 | 0.0691 | 23.2113 | 21.13 |

### Sample 3

| | | | | crucible + | % |
|---|---|---|---|---|---|
| | empty | crucible + | sample | sample after | mineral |
| Sample | crucible | sample | weight | ignition | fiber |
| Core | 22.5716 | 25.8667 | 3.2951 | 24.1851 | 48.97 |
| Ribs | 24.0274 | 24.0723 | 0.0449 | 24.0374 | 22.27 |

2. 4.0 mm×50 mm Compression Screws (CS)

### Sample 1

| | | | | crucible + | % |
|---|---|---|---|---|---|
| | empty | crucible + | sample | sample after | mineral |
| Sample | crucible | sample | weight | ignition | fiber |
| core | 24.6966 | 27.0025 | 2.3059 | 25.8151 | 48.5 |
| ribs | 21.2507 | 21.3263 | 0.0756 | 21.2659 | 20.1 |

### Sample 2

| | | | | crucible + | % |
|---|---|---|---|---|---|
| | empty | crucible + | sample | sample after | mineral |
| Sample | crucible | sample | weight | ignition | fiber |
| core | 23.6956 | 26.0148 | 2.3192 | 24.8326 | 49.03 |
| ribs | 20.8187 | 20.9041 | 0.0854 | 20.8374 | 21.90 |

Sample 3

| | | | | crucible + | % |
|---|---|---|---|---|---|
| | empty | crucible + | sample | sample after | mineral |
| Sample | crucible | sample | weight | ignition | fiber |
| core | 23.5976 | 25.9333 | 2.3387 | 24.7606 | 49.79 |
| ribs | 21.8769 | 21.9783 | 0.1014 | 21.8981 | 20.91 |

3. 2.4 mm×50 mm hexagonal nail

### Sample 1

| | | | | crucible + | % |
|---|---|---|---|---|---|
| | empty | crucible + | sample | sample after | mineral |
| Sample | crucible | sample | weight | ignition | fiber |
| core | 23.6997 | 24.8403 | 1.1406 | 24.2535 | 48.6 |
| ribs | 22.5756 | 22.6266 | 0.051 | 22.586 | 20.4 |

### Sample 2

| | | | | crucible + | % |
|---|---|---|---|---|---|
| | empty | crucible + | sample | sample after | mineral |
| Sample | crucible | sample | weight | ignition | fiber |
| core | 22.8427 | 23.9899 | 1.1472 | 23.4139 | 49.79 |
| ribs | 23.6006 | 23.6636 | 0.0630 | 23.6144 | 21.90 |

### Sample 3

| | | | | crucible + | % |
|---|---|---|---|---|---|
| | empty | crucible + | sample | sample after | mineral |
| Sample | crucible | sample | weight | ignition | fiber |
| core | 23.2012 | 24.3878 | 1.1866 | 23.7895 | 49.58 |
| ribs | 24.0289 | 24.0936 | 0.0647 | 24.0429 | 21.64 |

### Conclusions

The content of mineral filler in core and ribs was determined to three different mineral fiber reinforced biocomposite implants.

For the 4.0 mm×50 mm hexagonal nail:

The range of mineral content in core is 48.97-50.7% (w/w), with mean 49.7% (w/w).

The range of mineral content in ribs is 17.6-22.27% (w/w), mean 20.3%.

For 4.0 mm×50 mm Compression Screws:

The range of mineral content in core is 48.5-49.79% (w/w), mean 49.1% (w/w).

The range of mineral content in ribs is 20.1-21.9% (w/w), mean 21.0%.

For 2.4 mm×50 mm hexagonal nail:

The range of mineral content in core is 48.6-49.7% (w/w), mean 49.3% (w/w).

The range of mineral content in ribs is 20.4-21.9% (w/w), mean 21.3% (w/w).

In total, for all tested implants, the mineral content average in core is 49.4%, and in ribs is 20.9%.

### Example 2—Pull Out Force of Ribbed Nail

Below example describes pull-out testing of smooth 2.0 mm nails (core diameter 2.0 mm) and ribbed 2.4 mm nails (core diameter 2.0 mm). The ribbed design adds significant pull-out strength to the nails. As described above, the mineral content in the ribs of the 2.4 mm nail is significantly less than the mineral content in the core of the 2.4 mm nail.

### Materials & Methods

Two types of implants were produced using reinforced composite material.
1. 2.4 mm×50 mm ribbed hexagonal nail (FIG. 2A-FIG. 2H): Edge to edge core diameter of 2.0 mm and edge to edge rib diameter of 2.5 mm. (i.e. rib height of 0.25 mm).
2. 2.0 mm×70 mm smooth circular nail: Core diameter of 2.0 mm.

Three samples were prepared for each type of implant.

Material composite raw material was comprised of PLDLA 70/30 polymer reinforced with 50% w/w continuous mineral fibers. Mineral fibers composition was approximately Na₂O 14%, MgO 5.4%, CaO 9%, B₂O₃ 2.3%, P₂O₅ 1.5%, and SiO₂ 67.8% w/w. Testing samples were manufactured by compression molding of multiple layers of composite material into a mold.

Pull-out force testing was performed on each type of implant according to a modified version of the method defined in ASTM F2502-17. In short, for each nail:

A 2.4 mm tunnel bole was drilled into a 25 or 30 pcf Sawbone block to a depth of 25-35 mm. The implant was inserted 20 mm into the tunnel by tamping with a light mallet. The Sawbone construct was then clamped into the base of a TestResources® Single Column Test Machine, load cell: 500 N model 220 Frame-1505017-10F (Test Resources, Shakopee, MN, USA). The top part of the nail (the segment that was not inserted into the Sawbone) was then clamped into a grip attached to the load cell. A constant displacement of 5 mm/minute was applied on the test sample until failure occurs (i.e. measured load drops to 50% of its peak value). The peak load pull-out force was then recorded for that sample.

10 samples were tested for the 2.4 mm ribbed nail and 7 samples were tested for the 2.0 mm smooth nails.

### Results

2.4 mm ribbed hexagonal nail:

Average pull-out strength was: 203.04 [N] (STD 15.79 [N]).

| | | | d_hole | Hex rib | Hex rib | Hex rib | Hex rib D | Max. pull-out |
|---|---|---|---|---|---|---|---|---|
| Sample # | Part No. | Lot No. | [mm] | D1 [mm] | D2 [mm] | D3 [mm] | Average [mm] | force [N] |
| 1 | OF2012450S | RND00064 | 2.35 | 2.45 | 2.49 | 2.47 | 2.47 | 206.46 |
| 2 | OF2012450S | RND00064 | 2.35 | 2.49 | 2.45 | 2.47 | 2.47 | 186.90 |
| 3 | OF2012450S | RND00064 | 2.35 | 2.47 | 2.49 | 2.45 | 2.47 | 188.70 |
| 4 | OF2012450S | RND00064 | 2.35 | 2.46 | 2.49 | 2.48 | 2.48 | 198.88 |
| 5 | OF2012450S | RND00064 | 2.35 | 2.48 | 2.47 | 2.49 | 2.48 | 216.02 |
| 6 | OF2012450S | RND00064 | 2.35 | 2.48 | 2.45 | 2.46 | 2.46 | 193.00 |
| 7 | OF2012450S | RND00064 | 2.35 | 2.49 | 2.47 | 2.46 | 2.47 | 212.48 |
| 8 | OF2012450S | RND00064 | 2.35 | 2.50 | 2.48 | 2.45 | 2.48 | 180.70 |
| 9 | OF2012450S | RND00064 | 2.35 | 2.50 | 2.50 | 2.51 | 2.50 | 228.25 |
| 10 | OF2012450S | RND00064 | 2.35 | 2.49 | 2.52 | 2.50 | 2.50 | 219.00 |
| average | | | | 2.48 | 2.48 | 2.47 | 2.48 | 203.04 |
| std | | | | 0.02 | 0.02 | 0.02 | 0.01 | 15.79 |

2.0 mm smooth circular nail (also referred to as “pin”)

Average pull-out strength was: 104.04 [N] (STD 46.36 [N])

| | | Pin | pin | |
|---|---|---|---|---|
| | | Weight | Diameter | |
| Pin Number | PIN LOT | (gr) | (mm) | pull out |
| 1 | F16013-3 | 0.334 | 2.050 | 129.01 |
| 2 | F16013-3 | 0.349 | 2.040 | 183.29 |
| 3 | F16013-3 | 0.360 | 2.040 | 67.64 |
| 4 | F16013-3 | 0.351 | 2.07 | 98.58 |
| 5 | F16013-3 | 0.3583 | 2.04 | 128.93 |
| 6 | F16013-3 | 0.3593 | 2.05 | 70.75 |
| 7 | F16013-3 | 0.3485 | 2.04 | 50.08 |

### Conclusions

While both the 2.4 mm hexagonal ribbed nails and 2.0 mm smooth circular nails (pins) had core diameter of 2.0 mm, the pull out force required to displace the 2.4 mm nail from the sawbone tunnel was about double the force required for the 2.0 mm nail. This effect can be attributed to the deformation of the ribs as they are inserted into a bone tunnel, which is facilitated by their moderated mineral quantity.

It will be appreciated that various features of the invention which are, for clarity, described in the contexts of separate embodiments may also be provided in combination in a single embodiment. Conversely, various features of the invention which are, for brevity, described in the context of a single embodiment may also be provided separately or in any suitable sub-combination. Various sub-embodiments may be combined in various combinations, even if not explicitly described herein. It will also be appreciated by persons skilled in the art that the present invention is not limited by what has been particularly shown and described hereinabove.

All references cited or described herein are hereby incorporated by reference as if set forth herein to the extent necessary to support the description of the present invention and/or of the appended claims.

Although the invention has been described in conjunction with specific embodiments thereof, it is evident that many alternatives, modifications and variations will be apparent to those skilled in the art. Accordingly, it is intended to additionally embrace all such alternatives, modifications and variations that fall within the spirit and broad scope of the appended claims.

What is claimed is:

1. An implant comprising a plurality of bioabsorbable mineral fibers and polymer wherein the implant comprises a core, surface layer and one or more protrusions on the surface layer; wherein the mineral content of the protrusion is lower than the mineral content of the implant core; and wherein a height of the protrusions is in a ratio to the measured dimension of the implant, in a range of 1:40 to 40:1.

2. The implant of claim 1, wherein said ratio is in a range of 1:30 to 30:1, 1:20 to 20:1, 1:10 to 10:1; 1:8 to 8:1; a range of 1:5 to 5:1; a range of 1:4 to 4:1; a range of 1:3 to 3:1, a range of 1:2 to 2:1, or any integral value in between.

3. The implant of claim 1, wherein the relative height of the protrusions increases the pull out strength of the implant by at least 50% compared with an implant without protrusions.

4. The implant of claim 1, wherein the protrusions are between 0.05 mm and 2 mm, 0.1 mm and 1 mm or 0.2 and 0.8 mm in height.

5. The implant of claim 1, wherein mineral content of the protrusion as a percentage of the entire composition is 10-40, 15-35, or 20-35 percentage points lower than the mineral content of the implant body as a percentage of its entire composition.

6. The implant of claim 1, wherein the protrusions are in the shape of ribs, threads, hooks, quills, spikes, burrs or clips.

7. The implant of claim 6, wherein the protrusions are in the shape of ribs.

8. The implant of claim 1, wherein the cross-section of the protrusions are in the shape of circles or hexagons.

9. The implant of claim 1, wherein the protrusions are between 0.10 mm and 2 mm, 0.2 mm and 1 mm or 0.4 and 0.8 mm in width at the base where the protrusion meets the implant.

10. The implant of claim 1, wherein the protrusions are between 0.01 mm and 0.3 mm, 0.02 mm and 0.2 mm or 0.02 and 0.1 mm in width at the tip.

11. The implant of claim 1, wherein the length of the implant is between 10 mm and 200 mm, or 15 mm and 150 mm.

12. The implant of claim 1, wherein the implant diameter is between 1 mm and 14 mm, 1 mm and 10 mm, 1.5 mm and 6 mm or 2.0 mm and 4 mm.

13. The implant of claim 1, wherein the implant is cannulated.

14. The implant of claim 1, wherein said polymer surface is ablated to a controlled extent, such that a structure of said fibers is maintained upon ablation of said polymer surface; wherein the fiber structure is maintained, wherein at least 50, 65, 80, 85, 90, 95% of surface fibers retain their geometric structure.

15. The implant of claim 1, wherein the protrusions are unidirectional.

16. The implant of claim 1, wherein the protrusions are a combination of unidirectional and bidirectional.

17. The implant of claim 1, wherein said body composition comprises a biodegradable polymer; wherein said biodegradable polymer comprises a homopolymer or a copolymer; wherein said copolymer comprises a random copolymer, block copolymer, or graft copolymer; wherein said polymer comprises a linear polymer, a branched polymer, or a dendrimer, of natural or synthetic origin; wherein said polymer comprises lactide, glycolide, caprolactone, valerolactone, carbonates (e.g., trimethylene carbonate, tetramethylene carbonate), dioxanones (e.g., 1,4-dioxanone), 8-valerolactone, 1,dioxepanones (1,4-dioxepan-2-one and 1,5-dioxepan-2-one), ethylene glycol, ethylene oxide, esteramides, y-ydroxyvalerate, ˜-hydroxypropionate, alpha-hydroxy acid, hydroxybuterates, poly (ortho esters), hydroxy alkanoates, tyrosine carbonates, polyimide carbonates, polyimino carbonates such as poly (bisphenol A-iminocarbonate), poly (hydroquinone-iminocarbonate, (polyanhydrides, polymer drugs (e.g., polydiflunisol, polyaspirin, and protein therapeutics), sugars, starch, cellulose and cellulose derivatives, polysaccharides, collagen, chitosan, fibrin, hyaluronic acid, polypeptides, proteins, poly (amino acids), polylactides (PLA), poly-L-lactide (PLLA), poly-DL-lactide (PDLLA), polyglycolide (PGA), copolymers of glycolide, glycolide/trimethylene carbonate copolymers (PGA/TMC); other copolymers of PLA, such as lactide/tetramethylglycolide copolymers, lactide/trimethylene carbonate copolymers, lactide/d-valerolactone copolymers, lactide/£-caprolactone copolymers, L-lactide/DL-lactide copolymers, glycolide/L-lactide copolymers (PGA/PLLA), polylactide-co-glycolide; terpolymers of PLA, such as lactide/glycolide/trimethylene carbonate terpolymers, lactide/glycolide/ε-caprolactone terpolymers, PLA/polyethylene oxide copolymers; polydepsipeptides; unsymmetrically 3,6-substituted poly-I,4-dioxane-2,5-diones; polyhydroxyalkanoates; such as polyhydroxybutyrates (PHB), PHB/bhydroxyvalerate copolymers (PHB/PHV); poly-b-hydroxypropionate (PHPA); poly-p-dioxanone (PDS); poly-d-valerolactone poly-ε-capralactone, poly(ε-caprolactone-D-L-lactide) copolymers, methylmethacrylate-N-vinyl pyrrolidone copolymers; polyesteramides; polyesters of oxalic acid; polydihydropyrans; polyalkyl-2-cyanoacrylates; polyurethanes (PU); polyvinylalcohol (PV-A); polypeptides; poly-b-malic acid (PMLA): poly-b-alkanoic acids; polycarbonates; polyorthoesters; polyphosphates; poly(ester anhydrides); and mixtures thereof; and derivatives, copolymers and mixtures thereof.

18. The implant of claim 17, wherein the polymer is in a form of a polymer matrix; wherein said polymer matrix comprises a polymer selected from the group consisting of PLLA (poly-L-lactide), PDLLA (poly-DL-lactide), PLDLA, PGA (poly-glycolic acid), PLGA (poly-lactide-glycolic acid), PCL (Polycaprolactone), PLLA-PCL and a combination thereof.

19. The implant of claim 18, wherein if PLLA is used, the matrix comprises at least 30% 50%, or at least 70% PLLA.

20. The implant of claim 17, wherein if PLDLA is used, the matrix comprises at least 5%, at least 10%, or at least 20% PLDLA.

21. The implant of claim 1, wherein an inherent viscosity (IV) of the polymer matrix alone is in the range of 0.2-6 dl/g, 1.0 to 3.0 dl/g, 1.5 to 2.4 dl/g, or 1.6 to 2.0 dl/g, wherein IV is measured according to a flow time of a polymer solution through a narrow capillary relative to the flow time of the pure solvent through the capillary.

22. The implant of claim 1, wherein said mineral material of said body composition comprises ranges of the following elements, all mol %:
Na₂O: 11.0-19.0 mol %,
CaO: 9.0-14.0 mol %,
MgO: 1.5-8.0 mol %,
B₂O₃: 0.5-3.0 mol %,
Al₂O₃: 0-0.8 mol %,
P₂O₃: 0.1-0.8 mol %, and
SiO₂: 67-73 mol %.

23. The implant of any of the above claim 22, wherein said mineral material of said body composition comprises ranges of the following elements, all mol %:
Na₂O: 12.0-13.0 mol %,
CaO: 9.0-10.0 mol %,
MgO: 7.0-8.0 mol %,
B₂O₃: 1.4-2.0 mol %,
P₂O₃: 0.5-0.8 mol % and,
SiO₂: 68-70 mol %.

24. The implant of any of the above claim 23, wherein said mineral material of said body composition comprises ranges of the following elements, all mol %:
Na₂O: 11.0-19.0 mol %,
CaO: 8.0-14.0 mol %,
MgO: 2-8.0 mol %,
B₂O₃: 1-3.0 mol %,
Al₂O₃: 0-0.5 mol %,
P₂O₃: 1-2 mol % and,
SiO₂: 66-70% mol %.
